(19) United States
(12) Patent Application Publication
(10) Pub. No.: US 2017/0058300 A1
(43) Pub. Date: Mar. 2, 2017

(54) METHODS FOR THE DIGESTION OF SOLUBLE COMPONENTS ISOLATED FROM THE SPENT GRAINS OF A FERMENTATION PROCESS

(71) Applicants: Jennifer Aurandt, Brighton, MI (US); James R. Bleyer, Maumee, OH (US)

(72) Inventors: Jennifer Aurandt, Brighton, MI (US); James R. Bleyer, Maumee, OH (US)

(73) Assignee: Valicor, Inc., Dexter, MI (US)

(21) Appl. No.: 15/250,149

(22) Filed: Aug. 29, 2016

Related U.S. Application Data
(60) Provisional application No. 62/210,521, filed on Aug. 27, 2015.

(51) Int. Cl. C12P 5/02

(52) U.S. Cl. CPC C12P 5/023

(57) ABSTRACT

A method of processing spent grains by removing suspended solids from the spent grains to produce a stream low in suspended solids, directing the stream low in suspended solids to an anaerobic digester, converting at least some soluble compounds to biogas, and producing a biogas. A method of processing spent grain, by separating a first stream consisting of spent grains into a second stream and a third stream wherein the second stream contains a majority of suspended solids, separating the third stream into a fourth stream and a fifth stream wherein the fifth stream is lower in suspended solids than the fourth stream, directing the fifth stream to an anaerobic digester, and converting at least some organic compounds to a biogas. A method of fermenting a grain product.

### BACKGROUND OF THE INVENTION

1. Technical Field

The present invention relates generally to processes for the anaerobic digestion of short chain organic compounds isolated from the spent grains of a fermentation process and in particular relates to a process for the rapid digestion of soluble organic compounds to produce an enriched protein stream and lower the overall energy requirement and carbon footprint of a fermentation facility.

2. Background Art

The rising cost, environmental impact and the unstable supply of crude oil has driven the desire to find new, low cost, reliable, domestic supply of liquid motor fuels. Ethanol has proven to be a viable substitution for petroleum derived gasoline. It reduces harmful air pollutants, dependence on fossil fuels and carbon emissions. Ethanol is produced through the fermentation of sugars into alcohol by the yeast. These sugars can be derived from plants such as sugar cane or sugar beets. Alternatively, starches from grains can be hydrolyzed into sugars as fermented. Historically, corn has been the predominant grain used to produce ethanol, but other grains such as milo and wheat have also been used. The spent grain from the fermentation process is generally recovered as an animal feed. In the case of ethanol, the spent grain is generally referred to as Distiller's Grains.

Fermentation processes produce many other products, such as bio-chemicals and nutraceuticals. Xanthum gum is an example of a bio-chemical produced by the fermentation of carbohydrates by the bacteria Xanthomonas campestris. Many nutraceuticals are produced through fermentation processes utilizing bacteria, fungi, and algae.

Fermentation processes are also used to produce beverage alcohol including wine, whiskey, bourbon and beer. Some fermentation processes convert the starch in grains to sugars and the spent grains are removed prior to fermentation.

In the case of corn ethanol, corn is ground and mixed with water to produce a slurry. The slurry is heated and treated with enzymes to convert the starch to monomer sugars. Yeast convert the sugars in the slurry to carbon dioxide (CO₂) and alcohol, resulting in an intermediate product known as beer. The CO₂ is vented or recovered as a by-product.

The alcohol is removed from the beer in a stripping column. The stripping column bottoms, referred to as “whole stillage,” contain unfermentable components of the grain such as fiber, cereal proteins and lipids, yeast cells, unconverted starch and sugars, and secondary metabolites such as glycerol and organic acids.

Whole stillage is separated into a wet cake, also known as Wet Distiller's Grains (WDG), and thin stillage. A portion of the thin stillage is evaporated to produce a concentrate, sometimes referred to as distiller's solubles, or more commonly “syrup,” that can be sold and/or added to the wet cake to produce wet distiller's grains with solubles (WDGS). Distiller's oil can be removed from the thin stillage or syrup and recovered as a co-product. The wet cake with solubles can be sold as is but is typically dried to produce dried distiller's grain with solubles (DDGS). If syrup is not added to wet cake, the dried product is known as dry distiller's grains (DDG). Wet cake (WDG), WDGS, DDG, DDGS and distiller's corn oil are conventional distiller's products derived from stillage and are valuable animal feed products and are essential to the economic viability of the process.

The remaining thin stillage is recycled to the front end of the plant as mash water or commonly called “backset” and is integral to the efficient operation of the ethanol process. The recycling of the thin stillage is essential to maintaining a balance of water within the plant and allows the ethanol plant to operate as a zero water discharge. The thin stillage has beneficial components that improve the efficiency of hydrolysis and fermentation, such as minerals, residual enzymes and soluble protein. However, the thin stillage also has components that make it less than ideal for use as backset. The thin stillage contains unfermentable solids that can displace fresh source of starch, reducing titers. Thin stillage also contains glycerol, organic acids and other off product metabolites that are fermentation inhibitors.

Fermentation processes from time to time can experience upsets that result incomplete conversions. Such upsets can be the result of temperature excursions, bacterial contamination, poor yeast quality, and/or nutrient imbalance. Such upsets can result in higher than normal levels of inhibiting off product metabolites. As thin stillage is recycled as backset, additional fermentation batches are affected. Because of the high stillage recycle rate within an ethanol facility, it can take several fermentation cycles to recover from upsets.

The traditional ethanol production process as described can use high amounts of energy. A typical plant can use as much as 30,000 btu of natural gas and 1.0 kw*hr or electricity for every gallon of ethanol produced.

Anaerobic digestion is a process that converts organic matter into primarily methane and carbon dioxide and can provide multiple benefits to a fermentation process. Anaerobic digestion can remove the deleterious compounds from backset and provide biogas to offset the use of natural gas. The prior art discloses methods for the anaerobic digestion of fermentation stillage.

In U.S. Pat. No. 8,153,006, assigned to Procorp Enterprises LLC, Fessler et al. disclose a process for treating thin stillage from an ethanol production process by an anaerobic digester system equipped with an external solids/liquid separator such as an ultrafiltration (UF) membrane unit. Ammonia rich liquid permeate can be obtained from the UF unit and optionally recycled to the digester, recycled to the ethanol fermentation process in lieu of fresh water and ammonia or used to produce a fertilizer such as magnesium-ammonium-phosphate (“struvite”).

In U.S. Pat. No. 8,669,083, assigned to Eisenmann Corp., Veit et al. disclose a process for the anaerobic digestion of thin stillage (and optionally syrup), thereby producing biogas and a liquid effluent stream. Effluent from anaerobic digestion can be recycled as backset to the pre-treatment (i.e. liquefaction/saccharification) section of the fermentation plant and reduces the usual amount of thin stillage backset.

In European Patent Application EP 2581439 A1 as applied for by Agraferm Technologies AG, H. Freidman discloses a process for treatment of ethanol stillage comprising the steps of separating stillage by for example a decanting centrifuge, membrane filter unit, screw press, drum filter and/or drum screen, into a thin fraction and a thick fraction and separately digesting the fractions. Freidman discloses that the thin fraction can be digested much more quickly than the thick fraction and hence the thin fraction digester can be of much smaller volume. The thin fraction need not be devoid of suspended solids as the upflow digester specified by Freidman is designed without pore-containing materials or filters. Freidman discloses a downstream “nitrogen sink” system to remove ammonia as a gas from the digestate and use of said ammonia to enrich solid and liquid fertilizer co-products. Freidman further discloses that the purified water resulting from digestion can be returned to the “ethanol plant.” Freidman discloses that the thin fraction is characterized only by having a lesser dry weight content than the thick fraction.

In European Patent Application EP 1790732A1 as applied for by Prokop Invest AS and others, Prochazka et al. disclose the comprehensive use of ethanol production stillage to give multiple end products including dried stillage with low salt content, granulated sludge from anaerobic digestion, solid fertilizer as struvite, elementary sulfur and waste heat. Prochazka et al. disclose a two stage separation of solids from raw stillage. In the first stage cake is separated from “raw” stillage by decantation centrifugation. Residual particles, especially cereal proteins, are removed from the decanter centrate by a method such as air flotation, centrifugation, vacuum filtration or combinations thereof. Prochazka et al. disclose that the removal of residual solids protects the anaerobic biomass granules from disintegration and that the protein sludge removed in this step can be dewatered and combined with the first stage cake to increase the nitrogenous content of the final dry animal feed produced thereof. Liquid fractions from both stillage separation steps are blended and acidified under controlled conditions at pH ranging between 4.8 and 9.2. The resulting mixture is then treated anaerobically with granulated acetogenic and methanogenic bacteria. The accumulated granulated sludge is removed and stored for sale. The biogas is treated to remove sulfur and then used for energy production. From the digester liquid fraction, nitrogenous substances are removed by dosing magnesium chloride and phosphoric acid resulting in precipitation of struvite that is separated and removed as a high-quality fertilizer. The liquid fraction is subsequently taken to aerobic final treatment where sludge is separated. After having been thickened, the sludge can be used in agriculture. Acidifying bacteria produce extra cellular enzymes that reduce proteins to peptides and amino acids. The amino acids are further reduced to short chain acids and nitrogen compounds such as ammonia. The use of such bacteria will lower the overall protein content of substrate. Prochazka et al. disclose the use of acidifying bacteria and does not recognize the advantage of excluding the first stage of an anaerobic digestion system.

In U.S. Patent Application No. 2014/0065685, G. Rosenberger et al. disclose a process for the treatment of thin stillage from an ethanol fermentation process using an anaerobic membrane bioreactor. The membrane bioreactor produces a highly clarified permeate that can be recycled as backset to the fermentation process without contributing suspended solids which would otherwise necessitate a reduction in the fresh feedstock solids charged to the fermenter. Rosenberger et al. do not disclose the removal of suspended solids prior to digestion.

In U.S. Patent Application No. 2014/0134697A1 as applied for by DSM IP Assets B.V., H. L. Bihl et al. disclose the digestion of organic materials, including fermentation waste such as brewers spent grains, to biogas. The process is a two-stage process whereby in the first stage the organic material is heat treated to pasteurize and then enzymatically treated with proteases and/or lipases and/or cellulases which respectively digest proteins, lipids and complex carbohydrates. The effluent of the first stage is separated into a liquid and a washed solid fraction. The liquid fraction is fed to the second stage, an anaerobic digestion process to produce biogas. Bihl et al. disclose the digestion of protein and lipids and does not recognize a process designed to exclude these compounds from digestion.

In U.S. Pat. No. 8,017,365 Rein et al., disclose a “process resource production system” to convert an ethanol byproduct such as whole stillage, thin stillage and thin stillage solubles (i.e. thin stillage with suspended solids removed) to coproducts including an inorganic fertilizer such as struvite, and three products from anaerobic digestion: biogas, biosolids (an organic fertilizer) and a liquid stream suitable for treatment to produce recycle water. Rein et al. disclose an embodied two-step process in which high protein solids are first removed from thin stillage and then oil is removed by adjusting pH to approximately 6 and separating the oil by a density separator. The high protein solids removed from thin stillage can be combined with DWG to enhance protein content. Rein et al. also disclose that the anaerobic digester effluent (including biosolids) can be sent to the ethanol plant evaporators for thickening and that the thickened biosolids can be added to DWG.

In U.S. Patent Application Publication No. US2010/0196979 A1 as applied for by BBI International Inc., Birkmire et al. disclose a process for converting brewers spent grains and other brewery biomass streams into cellulosic ethanol and other products such as pelletized fuel, biogas (via anaerobic digestion) and livestock feed. Birkmire et al. disclose conversion of brewery biomass streams including spent grains by a process of cellulosic pretreatment, enzymatic hydrolysis, fermentation to ethanol and ethanol separation by distillation and dehydration. Residual solid slurry from fermentation is separated by centrifugation into wet cake and the liquid centrate. The centrate can be clarified to concentrated syrup (retentate) and clean water stream (permeate) via membranes, or anaerobically digested to produce biogas. Retentate syrup can be added to the wet cake and dried to produce an animal feed. It is disclosed that the purified water resulting from digestion can be returned to the “ethanol plant.”

Others have disclosed systems that utilize membranes or combinations of membranes and anaerobic digestion. In U.S. Pat. No. 7,267,774 assigned to NouVeau Inc. (USA), Peyton et al. disclose a potable water or beverage product obtained by treating still bottoms in an ethanol production facility by means of membrane pressure filtration (ultrafiltration, nanofiltration, and reverse osmosis) and anaerobic digestion. An objective of Peyton et al. is to capture the mineral and nutrient content of the fermentation process in the water or beverage product. Whole stillage can be separated by decanting centrifugation to remove large solids prior to UF-NF-RO filtration. Anaerobic digestion of combined solids from stillage and the concentrate streams from UF-NF-RO filtration produces a biogas with sufficient energy to power the pressurized filtration system. Also key to Peyton et al. is maintaining the stillage warm so as to preserve its pasteurized state.

Methods for the treatment of various wastewaters with a two phase digester have been disclosed in prior art. In U.S. Pat. No. 4,022,665, assigned to Institute of Gase Technology, Sambhunath Ghosh, et al., disclose an “improved two phase anaerobic digestion process in which an initial phase continually receives an organic feed for short detention times of less than two days under conditions which efficiently liquefy and breakdown the feed to lower molecular weight acids and other intermediates.” In such two phase digesters, the lower molecular weight acids and other intermediates are further converted to methane in the second phase of the digester.

There are many drawbacks from the processes and apparatuses of prior art for the digestion of spent grains or portions thereof from a fermentation process. Two stage anaerobic digestion has very low BOD destruction rates requiring large digestion reactors. A typical 55 MGPY corn ethanol plant would require digesters with a total volume of over 50M gallons. The high capital investment required for such a digester would not be economically feasible.

The two phase process indiscriminately degrades organic compounds; short chain organic compounds such as acids, and long chain organic compounds, such as proteins and lipids. Distiller's oil and protein co-products are important to the economic viability of the ethanol plant. The degradation of these products by digestion is not desirable. The prior art does not contemplate the use of a digester to selectively digest non-protein and non-lipid components. For example, in the previously described U.S. Pat. No. 8,017,365, Rein et al. disclose a method for producing “recyclable water” by the digestion of organic compounds, but do not recognize a process designed to selectively digest organic compounds resulting in an effluent with an increased protein content.

While some technologies of the prior art remove solids from the thin stillage, the prior art does not contemplate the advantages of using the low solids stream in a high rate/second stage digester.

The prior art does not contemplate the use of a high rate/second stage digester to digest primarily short carbon chain compounds while leaving long chain carbon compounds virtually un-degraded.

Therefore, there is a need for a high rate digester that degrades short chain organic compounds while leaving long chain organic compounds virtually un-degraded thereby concentrating said long chain carbon compounds. There is a need for a digester that removes non-protein components from a spent grains or a portion thereof to produce a new product high in protein and/or fat.

In accordance with the present invention, there is provided a new system and process for the treatment of a low suspended solids stream isolated from the spent grains of a fermentation process that further provides for the production of biogas, a liquid high protein concentrate, and an improved backset.

### SUMMARY OF THE INVENTION

The present invention provides for a method of processing spent grains by removing suspended solids from the spent grains to produce a stream low in suspended solids, directing the stream low in suspended solids to an anaerobic digester, converting at least some soluble compounds to biogas, and producing a biogas.

The present invention also provides for a method of processing spent grain, by separating a first stream consisting of spent grains into a second stream and a third stream wherein the second stream contains a majority of suspended solids, separating the third stream into a fourth stream and a fifth stream wherein the fifth stream is lower in suspended solids than the fourth stream, directing the fifth stream to an anaerobic digester, and converting at least some organic compounds to a biogas.

The present invention further provides for a method of fermenting a grain product, by mixing a grain product with a liquid to produce a slurry, adding a microorganism to the slurry to produce a product of fermentation, removing a product of fermentation from the slurry to produce spent grains, and directing at least of portion of the spent grains to a anaerobic digester.

### BRIEF DESCRIPTION OF THE DRAWINGS

Other advantages of the present invention are readily appreciated as the same becomes better understood by reference to the following detailed description when considered in connection with the accompanying drawings wherein:

FIG. 1 is a flowchart of a prior art corn ethanol fermentation process;

FIG. 2 is a flowchart of a prior art two stage anaerobic digestion process;

FIG. 3 is a flowchart of the second stage anaerobic digestion process of the present invention;

FIG. 4 is a flowchart of the second stage anaerobic digestion process of the present invention further including removing proteins, carbohydrates and fats/oils prior to digestion;

FIG. 5 is a flowchart of one embodiment of the present invention of an second stage anaerobic digester added to a corn ethanol fermentation process;

FIG. 6 is a flowchart a high rate digester of the present invention; and

FIG. 7 is a graph of biological methane potential.

### DETAILED DESCRIPTION OF THE INVENTION

The following detailed description of embodiments of the invention references the accompanying drawings. The embodiments are intended to describe aspects of the invention in sufficient detail to enable those skilled in the art to practice the invention. Other embodiments can be utilized and changes can be made without departing from the scope of the claims. The following detailed description is, therefore, not to be taken in a limiting sense. The scope of the present invention is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated or as will be readily apparent to those skilled in the art from the description. For example, a feature, structure, act, etc. described in one embodiment can also be included in other embodiments, but is not necessarily included. Thus, the present technology can include a variety of combinations and/or integrations of the embodiments described herein.

Certain terms used throughout this description are taken to have the meanings defined below.

“Spent grains” as used herein refers to a product stream of fermentation process in which the starch portion of grain has been converted to soluble fermentable sugars and carbohydrates by a cooking and/or enzymatic process. Said fermentable sugars and carbohydrates can be separated as a liquid from the starch-depleted grain prior to the fermentation step, for example to make beverage beer. Said fermentable sugars and carbohydrates can be fermented to alcohol prior to separation, for example to make industrial or fuel ethanol.

“Stillage” as used herein, refers to a cloudy liquid produced during fermentation that includes solids that are not fermentable, solubles, oils, organic acids, salts, proteins, and various other components.

“Whole stillage” as used herein, refers to a resultant product stream in which the primary products of grain fermentation, e.g. an alcohol, have been stripped from the stream and before the stream is acted upon by any other process.

“Thin stillage” as used herein, refers to a resultant product stream in which some or all of the insoluble solids have been removed from whole stillage. Insoluble solids can be removed from the whole stillage by centrifugation, filtration, settling or any other suitable mechanism.

“Wet cake” or “wet distiller's grains” (WDG) as used herein, refers to insoluble solids removed from whole stillage including the liquid and soluble solids that remain with the insoluble solids after separation.

“Distiller's grain co-products” as used herein refers to the category of fibrous or proteinaceous products which can be derived from fermentation spent grains. In the dry-grind fuel ethanol process, examples include wet cake (DWG), protein enriched DWG, DDG, DDGS. For clarity in the present specification, distiller's corn oil is not included in this category. In other fermentation process such as beverage alcohol, grain is also used as a source of sugar. Once the starch in the grain has been converted to sugar, the spent grain is typically removed from the process before fermentation. Spent grains from these operations are referred to by various names including “brewer's spent grain” (BSG) or “distiller's spent grain” (DSG). When used herein, the terms distiller's grain co-products and dried distiller's grains includes spent grains from any fermentation process, including those where the spent grains are removed prior to fermentation.

“Distillers Oil” as used herein refers to the oil from the grain feedstock recovered from a fermentation process, either prior to or after fermentation.

“High protein solids” as used herein, refers to a stillage fraction that contains a higher level of protein on a dry weight basis than whole stillage or spent grains.

“Fermentation” as used herein, refers to a biological process, either anaerobic or aerobic, in which suspended or immobilized microorganisms or cultured cells in a suitable media are used to produce metabolites and/or new biomass.

“Off product metabolites” as used herein, refers to metabolites produced during a fermentation process other than those products targeted for production by the fermentation process.

“Protein” as used herein, refers to organic molecules, which can be soluble or insoluble, including individual amino acids, short and long peptide chains, or proteins.

“Protein depleted stream” as used herein, refers to a stream in which some or all of the protein has been removed.

“Low protein stream” as used herein, refers to a stream that has a lower protein content, including no protein, than the stream from which it was extracted.

“High protein stream” as used herein, refers to a stream that has higher protein content than the stream from which it was extracted.

“Non-protein component(s)” as used herein, refers to soluble or in-soluble solids including fiber, simple and complex carbohydrates, lipids, phospholipids, nucleic acids, organic acids, alcohols, inorganic minerals and salts and excludes those components described as “protein” above.

“Second stage” as used herein, refers to a method of digesting short chain organic compounds in an anaerobic process, said process utilizing primarily acetogenic and methanogenic bacteria.

“Bio-solids” as used herein, refers to solids recovered from anaerobic digester comprising microorganisms of the digestion process.

In accordance with the present invention, described herein is a system and process for the treatment of a low suspended solids stream isolated from the spent grains of a fermentation process that enables the extraction of multiple co-products and further provides for the production of biogas, an enriched protein stream, a liquid high protein concentrate and an improved backset.

Most generally, the present invention provides for a method of producing a biogas from a liquid stream isolated from spent grains by processing in a second phase anaerobic digester thereby concentrating the oil and/or protein content of the liquid stream. More specifically, a method of processing spent grains is provided by removing suspended solids from the spent grains to produce a stream low in suspended solids, directing the stream low in suspended solids to an anaerobic digester, converting at least some soluble compounds, and producing a biogas.

Anaerobic digesters utilize groups of bacteria to reduce complex macromolecules into methane and carbon dioxide. Anaerobic digester typically consist of four phases; hydrolysis, acidogenisis, acetogenisis and methanogenisis. The four phases are grouped in to two stages—hydrolysis and acidogenisis are collectively referred to as the first stage or “acid” stage, and acetogenisis and methanogenisis are collectively referred to as the second stage or “methane” stage. In the first phase, hydrolytic bacteria secrete enzymes which breakdown macromolecules such as protein, lipids, and carbohydrates into soluble molecules with smaller atomic masses, such as peptides, fatty acids and sugars. These soluble molecules are absorbed by the acidogenic bacteria which produces simple molecules with low molecular weight, such as short chain organic acids, alcohols, hydrogen and carbon dioxide. In the third phase, the products of the acidogenisis are reduced to acetic acid, hydrogen and carbon dioxide by acetogenic bacteria. Methanogens, the organisms of the fourth phase, convert the products of the third phases into methane. The result of the anaerobic digester process is a biogas rich in methane and carbon dioxide and bio solids rich in bacteria and organic matter. The four phases of anaerobic digestion do not have the same kinetics. The conversion of the large macromolecules of the first phase is a slow process and can take 30 days or more. The kinetics of the third and fourth phase much higher and conversion can take as little as 12-24 hours.

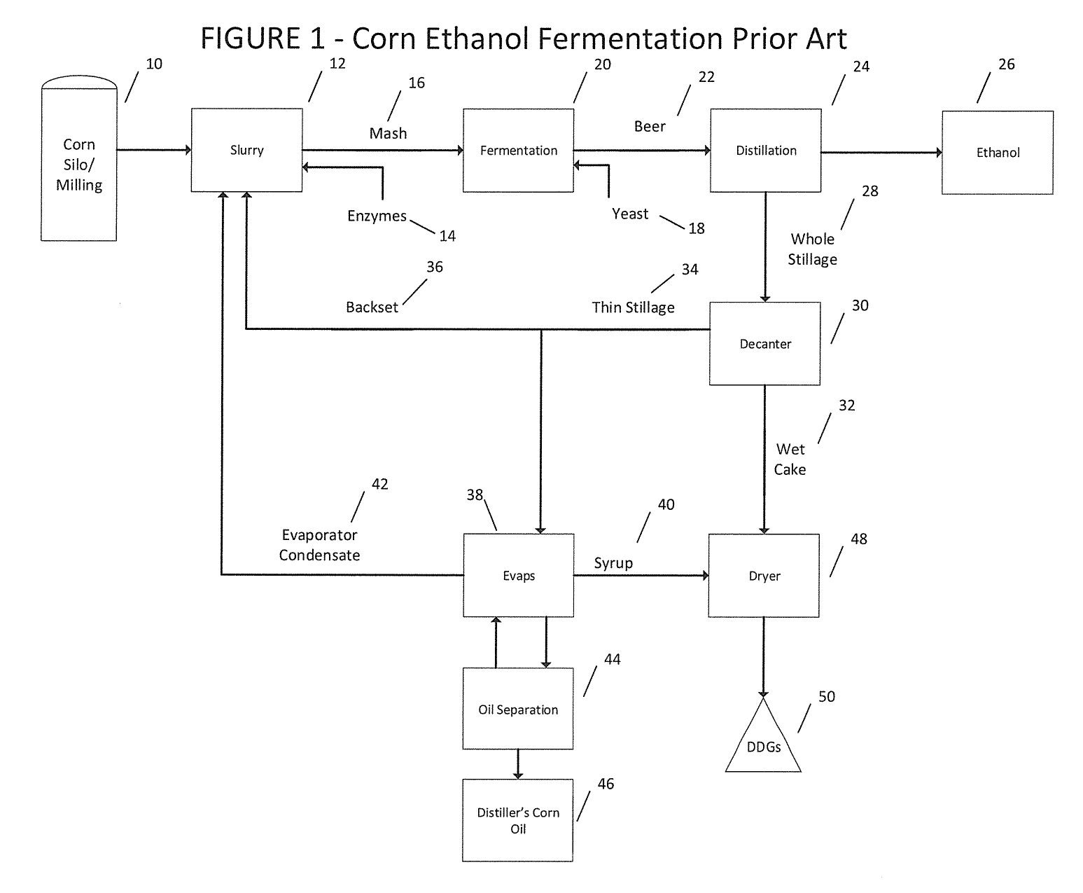

As a representative of a grain fermentation process, a dry-grind ethanol process is depicted in FIG. 1 (labeled Corn Ethanol Fermentation Prior Art). In the dry-grind process, whole grain is milled to flour (10) and slurried (12). The slurry is treated with one or more enzymes (14) to convert the starch in the slurry to sugars creating a fermentation mash (16). An organism such as yeast (18) is added to the mash to convert (20) the sugars to beer (22). The ethanol (26) is stripped from the slurry in a distillation column (24) to produce whole stillage (28). Whole stillage is recovered and separated into wet cake (32) and thin stillage (34). In U.S. dry-grind ethanol plants, the decanting centrifuge (30) is the most common whole stillage separation device although any suitable solid-liquid separation mechanism, including, but not limited to centrifuge, filtering centrifuge, vibrating screen, pressure screen, paddle screen, filter, and membrane or combinations thereof can be applied. A portion of the thin stillage known as backset (36) is recycled to the front end of the plant as make-up water for slurrying fresh grain. The balance of the thin is evaporated to syrup (40) in a multi-effect evaporator (38). Evaporator overheads are condensed to evaporator condensate (42) and used at the front end of the plant as additional make-up water. Grain oil is commonly recovered from the concentrated thin stillage by centrifugation (44) at an intermediate stage of evaporation. In the case of corn, this oil is commonly referred to as Distiller's Corn Oil (46). Various chemicals such as demulsifiers can be added to enhance oil separation. Syrup from the last stage of evaporation can be sold as is but more commonly it is added to wet cake and sold either wet as wet distiller's grains with solubles (WDGS), or most commonly, dried (48) to produce DDGS (50) having less than 15% moisture. The spent grains and/or products derived from spent grains can be directed to an anaerobic digester process of the present invention.

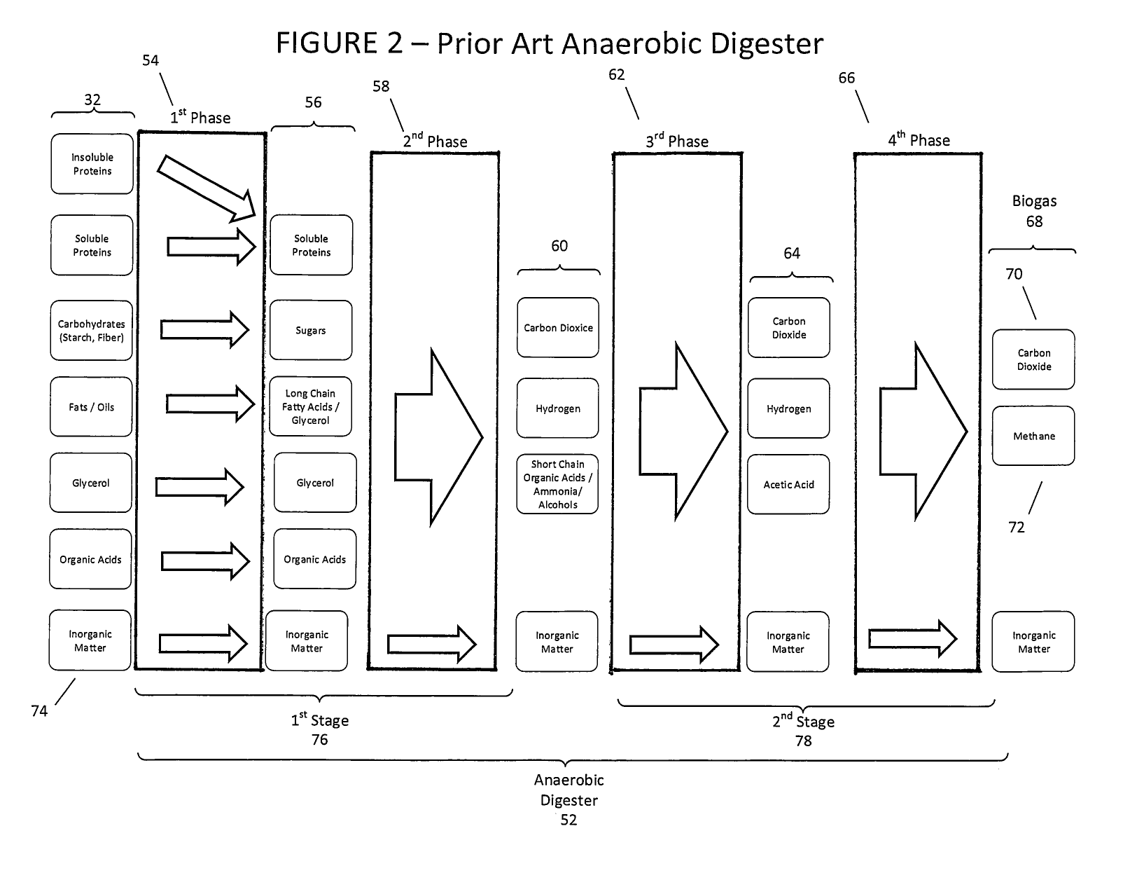

Specifically examining the flow of stillage through digesters of the prior art and comparing to embodiments of the present invention, one can recognize the utility and value of the present invention. Referencing FIG. 2 labeled “Prior Art Anaerobic Digester”, thin stillage (32), isolated from the spent grains of a fermentation process and including insoluble protein, soluble protein, oil, carbohydrates, such as starch and fiber, and inorganic material, such as minerals, is fed to an anaerobic digester (52). In the first phase (54), the components of the feed are hydrolyzed; insoluble protein to soluble protein, oil into long chain fatty acids and glycerol, carbohydrates into sugars (56). In the second phase (58), the products of the first phase (56) are converted primarily to carbon dioxide, hydrogen, ammonia, short chain organic acids and alcohols (60). In the third phase (62), the products of the second phase (60) are converted primarily to carbon dioxide, hydrogen and acetic acid (64). In the fourth phase (66), the products of the third phase (64) are converted to a biogas (68) comprising primarily carbon dioxide (70) and methane (72). Inorganics (74) are not converted in any of the phases and pass through the digester. The first phase and second phase are referred to collectively as the first stage (76). The third phase and fourth phase are referred to collectively as the second stage (78)

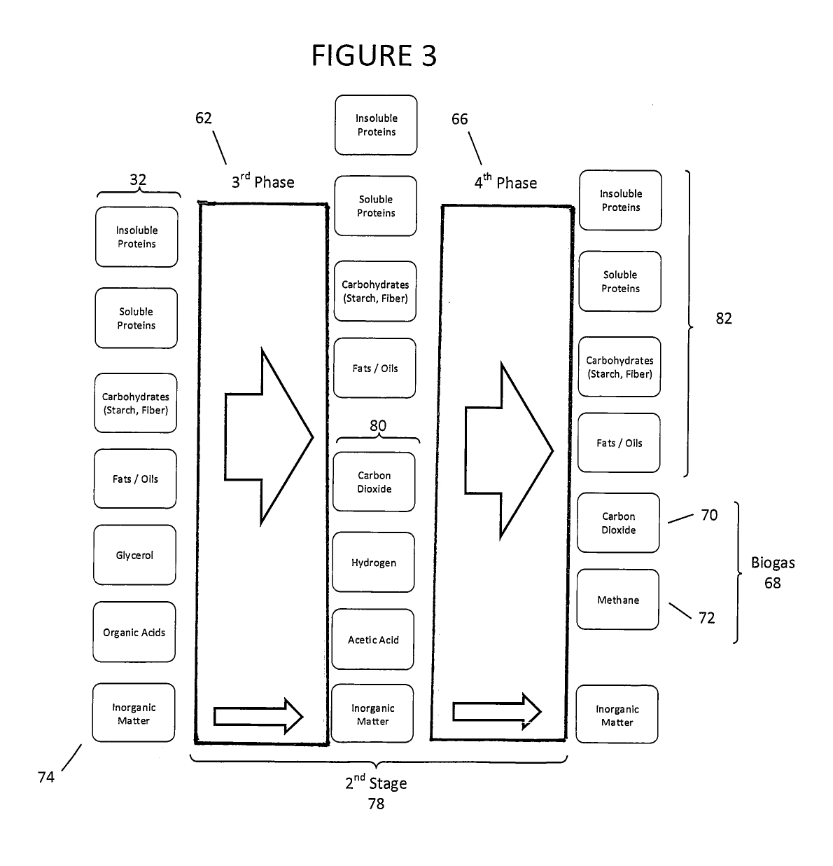

FIG. 3 illustrates an embodiment of the present invention. Thin stillage (32) isolated from the spent grains of a fermentation process and including insoluble protein, soluble protein, oil, carbohydrates, such as starch and fiber, and inorganic material, such as minerals, is fed directly to the second stage (78) of an anaerobic digester consisting of a third phase (62) and fourth phase (66). The organic acids and glycerol are converted to acetic acid, hydrogen and carbon dioxide (80). The products of the third phase (80) are converted to biogas (68) comprising primarily methane (72) and carbon dioxide (70) by bacteria of the fourth phase (66). Bacteria of the second stage are unable to convert the complex macromolecules of proteins and oils and they pass through the digester (82).

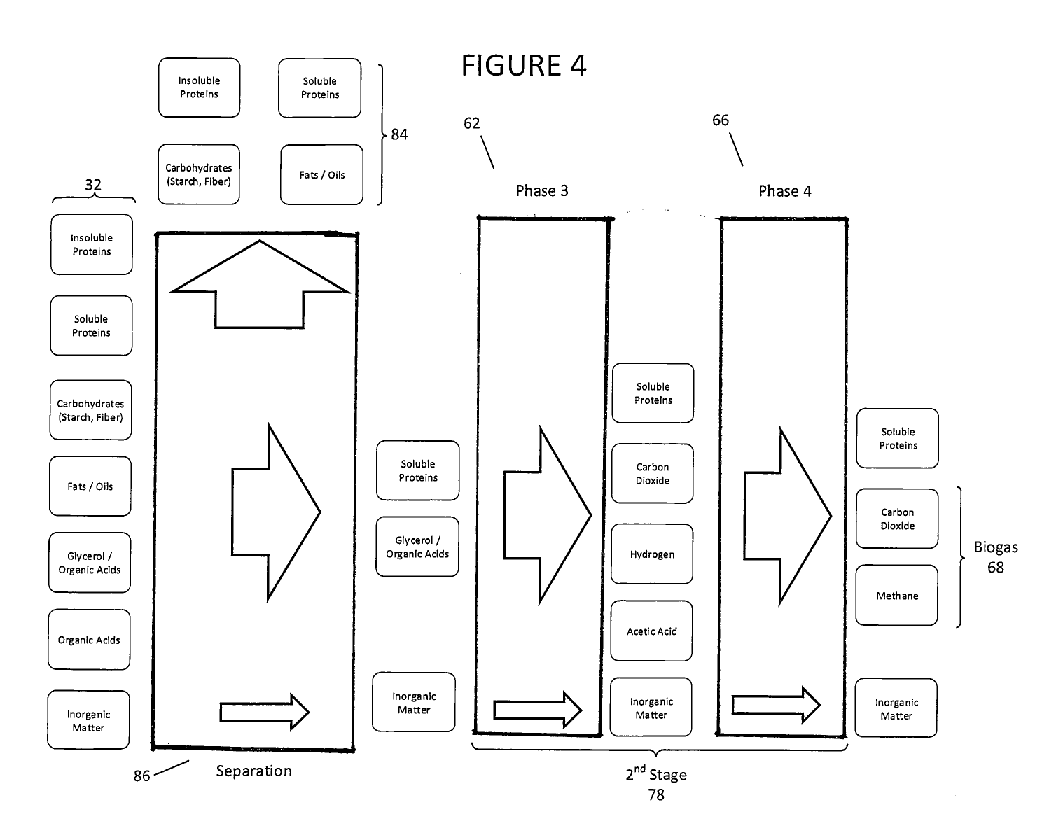

FIG. 4 illustrates an embodiment of the present invention. Prior to second stage of an anaerobic digester (78), suspended solids (84) comprising insoluble protein, carbohydrates and oils are separated (86) from the thin stillage.

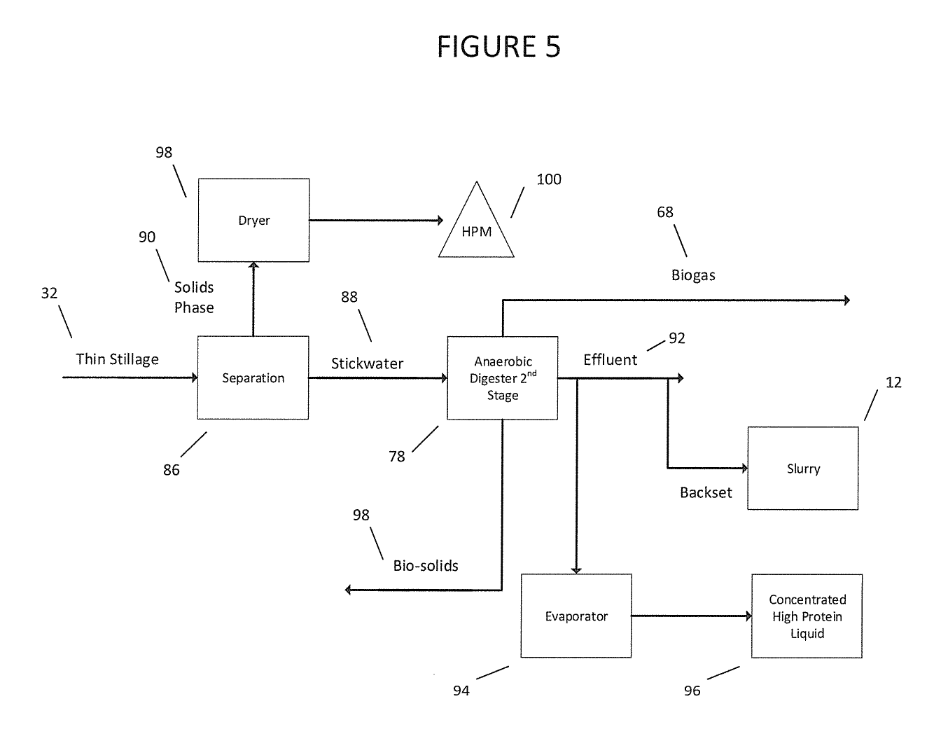

FIG. 5 illustrates an exemplary embodiment of the present invention. Suspended solids are separated (86) from thin stillage (32) to produce a low suspended solids stream, stickwater (88) and a solids phase (90). Stickwater is fed to a second stage anaerobic digester (78) where the organic acids and glycerol are digested and converted to biogas (68). The effluent (92) from the anaerobic digester is collected and a portion is returned to the front end of a fermentation process (12). Water is removed from a portion of the effluent in, for example, an evaporator (94) to produce a concentrated high protein liquid (96). Bio-solids (98) comprising microorganisms are removed from the digester and collected for further use.

There are a number of methods to improve upon traditional digestion processes and such improvements are part of the present invention.

The present invention provides for the treatment of stillage from a fermentation process to increase its suitability as backset. Proteins in the stillage are desirable as they improve the health and function of microorganisms such as yeast. However, organic acids and glycerol can act as inhibitors, reducing the productivity of microorganisms. Typically, solids in backset cannot be fermented and if removed can be replaced with fermentable sugars or starch. As a result of adding more fermentable components, titers can be increased.

Therefore, in one embodiment of the present invention, stillage is directed to a second phase digester where at least one of the following organic compounds are converted to a biogas: organic acids, glycerol. In another embodiment of the present invention, at least part of the effluent is used as at least part of a fermentation media.

Distiller's grain products are used primarily as animal feeds and are desirable for their protein, fat and fiber content. Syrup is another co-product of grain fermentation that can be sold as an animal feed, but is low in value because of its undesirable nutritional profile. Syrup is produced by evaporating thin stillage in, for example, a multi-effect evaporator. Water and some of the low boiling organic acids volatilize in the evaporator, and are collected and condensed into evaporator condensate. The balance of the organic acids, glycerol and other components are concentrated into syrup.

The present invention provides for the treatment of spent grains or portions thereof to provide an additional co-product, such as animal feed. The methods of the present invention will selectively digest glycerol, organic acids and other short chain carbon compounds. Other longer chain compounds, such as peptides, amino acids, fatty acids and lipids are not digested. By selectively digesting primarily non-protein, non-fat components, the concentration of protein and/or fat will increase.

In one embodiment of the present invention, influent consisting of spent grains, portions thereof, or a stream isolated from spent grains is directed to a digester comprising primarily acetogenic and methanogenic organisms where primarily non-protein, non-oil organic compounds are converted to a biogas. The effluent can be recovered from the process. In another embodiment, the effluent can be evaporated to form syrup, recycled to the front end of the ethanol process as backset, or used as make-up water for other processes, such as boiler feed water or cooling tower make-up water. In another embodiment, the effluent can be further processed in an aerobic treatment process, sold as a co-product of the process, discharged to surface water, discharged to a treatment facility, land applied as, for example, a fertilizer or combinations thereof. Therefore, in one embodiment of the present invention, short chain carbon compounds are digested in an anaerobic process. In another embodiment, the digester microorganism community is comprised primarily of acetogenic and methanogenic microorganism. In another embodiment of the invention, the concentration of one or more organic compounds is reduced by at least 50%, the organic compounds can include acetic acid, lactic acid, other organic acids, ethyl alcohol, other alcohols, glycerol or combinations thereof. In another embodiment, the digestate or effluent of the digester is evaporated, recycled to another process, aerobically digested, and recovered as a co-product, evaporated, discharged, land applied or combinations thereof.

The present invention provides for the treatment of thin stillage to produce a product with an improved nutritional profile. In one embodiment of the present invention, the effluent recovered from the digestion process has a higher protein concentration, as measured by weight of dry matter, as compared to the feed to the digester. In another embodiment of the present invention, the protein content of the effluent is over 15% and more preferably over 19% an measured by weight of dry matter. In another embodiment of the present invention, the effluent recovered from the digestion process has a higher lipid content, as measured by weight of dry matter, as compared to the feed to the digester. In another embodiment of the present invention, one or more of the soluble compounds can be removed prior to or after the digester by any suitable means, including but not limited to precipitation, membrane filtration, or ion exchange. In another embodiment of the present invention, the recovered compounds are further concentrated by, for example, evaporation. For example, protein can be precipitated from the effluent.

In one embodiment of the present invention, stillage is directed to a second phase digester where at least one of the following organic compounds are converted to a biogas: organic acids and glycerol, and recovering an effluent with a higher protein content, fat content, or combinations thereof as compared to the digester feed. In one embodiment of the present invention, the stillage is evaporated prior to digestion. In another embodiment of the present invention, water is removed from the effluent. In another embodiment of the present invention, water is removed by evaporation. In another embodiment of the present invention, a protein product, a fat product or a pro-fat product is recovered. In another embodiment of the present invention, the recovered product is further dried to a moisture content of about 10%. In another embodiment of the present invention, the effluent is concentrated to about 25% or more solids by weight of dry matter by any suitable means, including, but not limited to evaporation. In another embodiment of the present invention the recovered product is added to another co-product of the fermentation process. In one embodiment of the present invention, the effluent can have high levels of protein, low levels of glycerol, low levels of organic acids or combinations thereof. In another embodiment of the present invention, grain is ground to a flour, slurried, treated with one or more enzymes to hydrolyze at least one component of the grain, fermented, separated into a product of fermentation and a spent grain, at least a portion of the spent grain is directed to a second stage digester where at least one short chain organic compound is removed, and an effluent of the digester is collected.

A second stage digester is characterized by high BOD destruction and short hydraulic retention times (HRT). The destruction rates are much faster than the ability of the microorganisms to reproduce. The design of the second stage digester must be such that the solids retention time is greater than the HRT. This can be achieved by methods including, but not limited to settling the microorganisms within the digester or separating the microorganisms from the effluent and recycling the microorganisms back to the digester. Such digester can include aspects of other high rate digesters including upflow anaerobic sludge blanket (UASB), anaerobic fixed film, and suspended media digesters. The HRT can be about 24 hours or less, and more preferably, about 12 hours or less.

Solids and oils can interfere in the operation of a high rate digester. The suspended solids of the influent interfere with the settling of the microorganisms. The suspended solids can displace or “wash out” the microorganisms causing the treatment zone to become depleted of the microoganisms. The removal of suspended solids also provides for the use of a smaller treatment reactor.

The present invention provides for the removal of components from spent grains that interfere with the operation of a high rate digester. In one embodiment, the influent to the high rate digester has a suspended solids content of about 2,000 ppm and more preferably about 1,000 ppm or less. In another embodiment, the influent to the high rate digester has an oil content of about 1% or less. In another embodiment, the removal of suspended solids can be accomplished in one or more steps by any suitable means, including but not limited to decanting centrifuge, disc stack centrifuge, nozzle disk centrifuge, filtration centrifuge, pressure screen, vibratory screen, static screen, wedge wire screen, paddle screen, filtration, membrane, dissolved air flotation or any suitable means. In another embodiment, the oil can be removed in one or more steps by any suitable means, including but not limited to decanting centrifuge, disc stack centrifuge, nozzle disk centrifuge, filtration centrifuge, pressure screen, vibratory screen, static screen, wedge wire screen, paddle screen, filtration, membrane, dissolved air flotation.

The suspended solids removed from the spent grains can be high in protein and/or lipids and can be further processed and recovered as co-products. Various methods can be used to influence the yield and concentration of the recovered protein and/or lipids. For example, whole stillage can be screened, filtered, sieved to harvest additional and higher concentration of protein. Stillage can be subjected to various pre-treatments, such as acid treatment, enzymatic hydrolysis, hydrothermal treatment, shearing, and/or grinding.

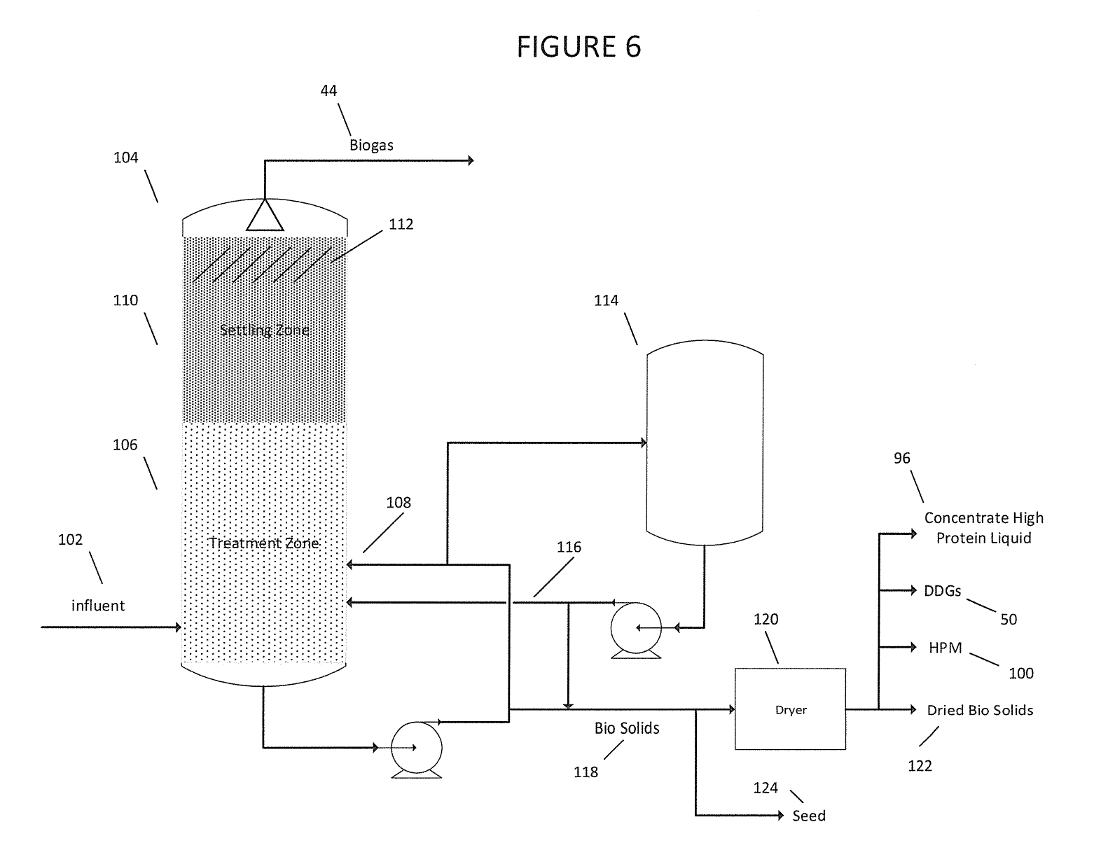

Referring to FIG. 6, influent (102) is directed to a treatment reactor (104) and is mixed with microorganisms in a treatment zone (106) by circulating the liquid in the reactor (108). Mixing can also be accomplished by any other suitable means, including agitation. The mixture is hydraulically conveyed to a settling zone (110) where the microorganisms separate from the mixture and return to the treatment zone. Such separation can be through quiescent decantation. The separation can be aided with the addition of lamella plates (112). The treatment zone and separation zone can be accomplished in one or more reactors. For example, the mixture from the treatment reactor can be directed to a second reactor (114) where the mixture is allowed to separate. The settled liquor will have high levels of the microorganism and can be recycled to the first reactor (116). The bio-solids (118) comprising microorganisms from the digestion process can be recovered, dried, for example by a dryer (120), to produce a dried bio-solids (122), added to distiller grain products, including but not limited to, HPM (100), DDGs (50) or concentrated high protein liquid (96), or used or sold as, for example, a seed for other digestion processes (124) or combinations thereof. Biogas (60) from the process is collected and can be used for any suitable purpose.

Therefore, in one embodiment of the present invention, a first stream including the spent grains from a fermentation process is separated into a second stream and a third stream wherein the third stream has a suspended solids content of about 2,000 ppm or less and more preferably about 1,000 ppm or less. In another embodiment of the present invention, the third stream has an oil content of 1% or less. In another embodiment of the present invention, the third stream is fed to an anaerobic digester. In another embodiment of the present invention the anaerobic digester is operated in a manner to promote a high rate of BOD destruction.

The separation of suspended solids can be accomplished in two or more steps. Therefore, in one embodiment of the present invention, spent grains are separated into a second stream and a third stream, the second stream containing a majority of the suspended solids. The third stream can be separated into a fourth stream and a fifth stream, the fifth stream containing about 2,000 ppm or less of suspended solids. In another embodiment, the fourth stream containing a protein content higher than the third stream and can be collected and processed to produce a high protein meal. Methods of the present invention produce a biogas. In one embodiment of the present invention, the biogas is recovered, used as a fuel source, used as a carbon source, separated into a stream rich in methane, separated into a stream rich in carbon dioxide, treated to remove undesirable compounds or combinations thereof.

Therefore, the present invention also provides for a method of processing spent grain, by separating a first stream consisting of spent grains into a second stream and a third stream wherein the second stream contains a majority of suspended solids, separating the third stream into a fourth stream and a fifth stream wherein the fifth stream is lower in suspended solids than the fourth stream, directing the fifth stream to an anaerobic digester, and converting at least some organic compounds to a biogas. This method can further include removing additional soluble compounds from the second stream by combing the second stream with at least one diluent. This method can further include removing additional soluble compounds from the fourth stream by combing the fourth stream with at least one diluent. Suspended solids can also be recovered from the fourth stream.

The present invention also provides for a method of fermenting a grain product by mixing a grain product with a liquid to produce a slurry, adding a microorganism to the slurry to produce a product of fermentation, removing a product of fermentation from the slurry to produce spent grains, and directing at least of portion of the spent grains to an anaerobic digester. Each of these steps has been described above. The anaerobic digester can contain microorganisms primarily classified as acetogenic or methanogenic. The concentration of one or more organic compounds can be reduced by about 50% or more, with the organic compounds selected from acetic acid, lactic acid, other organic acids, ethyl alcohol, other alcohols, glycerol, and combinations thereof. The method can further include the step of using effluent of the anaerobic digester as a fermentation media. The method can further include the step of recycling effluent of the anaerobic digester to a fermentation process. Effluent of the anaerobic digester can be recovered. The recovered effluent can be higher in protein that influent to the anaerobic digester. The method can further include at least one step of dehydrating the effluent, drying the effluent, and combinations thereof. The method can further include the step of concentrating the effluent. The method can also further include the step of adding at least some of the effluent to at least some of the spent grains. The method can further include the step of recovering bio-solids from the anaerobic digester. The method can further include at least one step chosen from dewatering the bio-solids, drying the bio-solids, and adding the bio-solids to the spent grains of a fermentation process or portions thereof.

The present invention also provides for a method of processing spent grains further including removing distiller's oil from one or more of the streams prior to anaerobic digestion.

The present invention also provides for a method of producing an improved backset including the steps of removing suspended solids, oil, or combinations thereof from spent grains or a portion thereof; directing the resultant stream to an anaerobic digester to remove at least some of the dissolved solids and directing the effluent of the anaerobic digester to a fermentation process.

The present invention also provides for a method of producing an improved product by directing an influent to a second stage anaerobic digester where the influent is comprised of spent grains of a fermentation process or portions thereof, digesting at least some of the organic compounds contained in the influent, and collecting a effluent where the protein and/or fat content of the effluent is higher than the influent. The present invention further provides for combining microorganisms from the digester with at least some of the effluent or portions thereof; dehydrating the effluent, concentrating the solids of the effluent, drying the effluent, or combinations thereof. The present invention further provides for combining microorganisms from the digester with at least one co-product of a fermentation process, including, but not limited to, distiller's grain product, WDG, DDGs, syrup, HPM, or concentrated high protein liquid.

The present invention provides for a method of increasing the protein concentration of a stream isolated from the spent grains of a fermentation process by converting non-protein components in an anaerobic digester. The present invention further provides for the removal of water from the high protein liquid feed to further concentrate the protein.

### Example 1

### Anaerobic Digestion of Stickwater

Procedures

For the present EXAMPLE 1, whole stillage obtained from a commercial ethanol plant was filtered through a 600 micron pan filter. The filtrate and retentate were collected. The filtrate was heated to 250° F. and held at that temperature for 40 minutes, and then cooled to 180° F. The filtrate was then centrifuged to separate the filtrate into a high protein stream and a low protein stream. The pH adjusted low protein stream with nutrients was fed to a 15 L Up-Flow Anaerobic Sludge bed reactor (UASB) seeded with bacteria obtained from an operating anaerobic digester of a commercial ethanol plant and used for treatment of evaporator condensate. The bacterial consortium consists of second phase anaerobic digestion organisms, primarily acetogens and methanogens. The reactor was operated with a hydraulic residence time (HRT) of 12 hours and a bed solids content of 6%. Samples of the low protein stream were collected before feeding the reactor and the effluent was sampled after passing out of the reactor system. Feed and effluent samples were analyzed by HPLC for total solids, glycerol, organic acids and protein content.

Results and Discussion

TABLE 1 shows the analysis of the low protein feed stream and effluent from the anaerobic digester system at steady state.

TABLE 1

| COMPONENT | MEASURED CHANGE |
|---|---|
| Glycerol | 95% Degradation |
| Organic Acids | 75% Degradation |
| Protein | Feed: 11% |
| (wt % on dry basis) | Effluent: 19% |

Organic acids and glycerol were effectively degraded by the anaerobic bacteria to create biogas. When those components were removed from the process stream, the concentration of protein increased in the low protein stream from 11% to 19% (dry basis).

### Example 2

### Small Scale Reactor Operation

Procedures

For the present EXAMPLE 2, filtrate was prepared as described in EXAMPLE 1. Two-three liter reactors were seeded with two liters of 6% solid material from a second stage anaerobic digester obtained from a dry grind ethanol plant. The reactors were consistently stirred and the effluent is pumped out at the rate equal to the influent. The reactors were operated at a 5 day 37° C. Biogas production measured using wet tip meter. A complete nutrient mix was added to the reactor in addition to potassium bicarbonate. Biogas samples were collected once a week to analyze for methane and carbon dioxide. The reactors we operated for one month.

Results and Discussion

The reactors produced 10.4 L biogas per liter of stickwater on average over the course of the month at an approximate 5 day HRT.

### Example 3

### Biological Methane Potential

Procedures

For the present EXAMPLE 3, filtrate was prepared as in EXAMPLE 1. An AMPTS Biomethane Potential (BMP) Test System (Bioprocess Control) was used to characterize filtrates viability as a substrate. A series of three 500 ml bottle replicates were loaded with a 2:1 VS (w/w) seed to substrate ratio to a total of 400 g and placed in a 37° C. water bath with stirring throughout the experiment. A triplicate set of bottles were loaded with the same seed, obtained from a second stage reactor operating at a dry grind corn ethanol plant, to use as a control. The gas was passed through 3M NaOH solution to remove CO2 from the produced biogas. Remaining gas, primarily methane, enters a calibrated flow cell which records the gas production in real time. The BMP reactors were operated for 30 days.

Results and Discussion

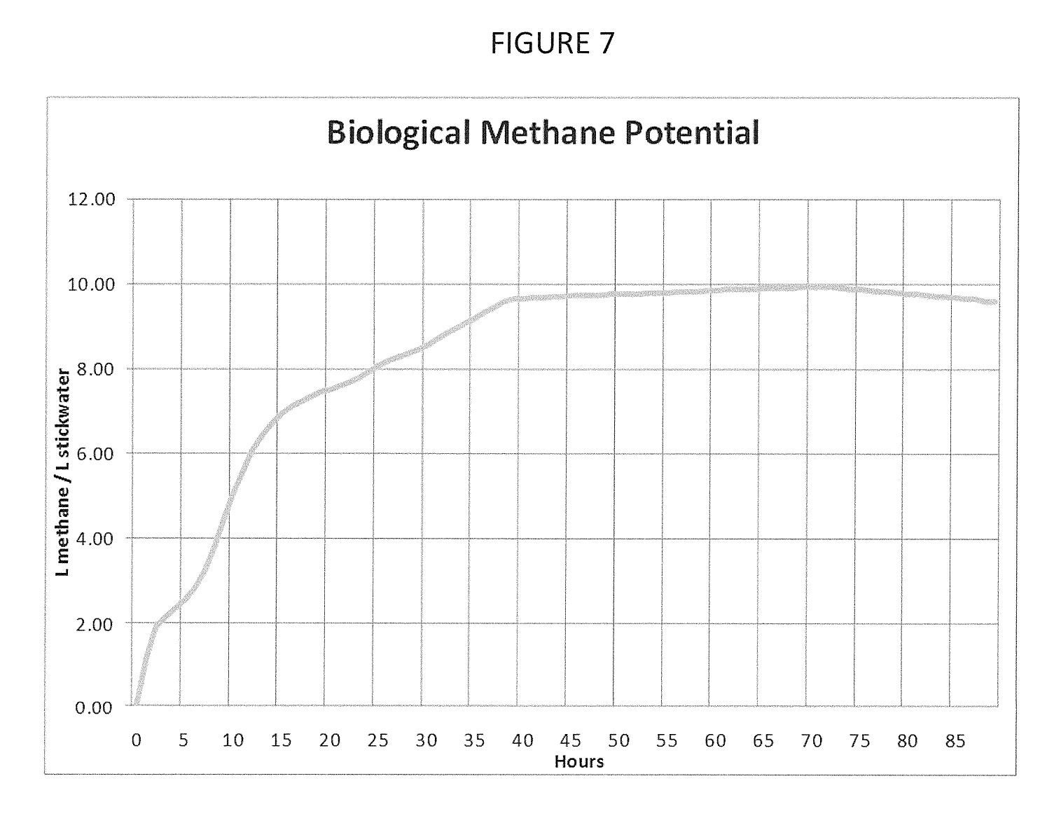

Referring to FIG. 7, the methane production reached a maximum of 10 liters of biogas per liter of stickwater. The theoretical production of methane from stickwater is 12 liters per liter of stickwater. The BMP obtained about 90% of theoretical methane production.

What is claimed is:

1. A method of processing spent grains, including the steps of:
removing suspended solids from the spent grains to produce a stream low in suspended solids;
directing the stream low in suspended solids to an anaerobic digester;
converting at least some soluble compounds to a biogas; and
producing a biogas.

2. The method of claim 1, wherein said removing step is performed in one or more steps by a process chosen from the group consisting of centrifugation, filtration, dissolved air flotation, decantation and combinations thereof.

3. The method of claim 1, where the stream low in suspended solids has a suspended solids level of about 2,000 ppm or less.

4. The method of claim 1, wherein the stream low in suspended solids has a suspended solids level of about 1,000 ppm or less.

5. The method of claim 1, wherein the anaerobic digester contains microorganisms primarily classified chosen from a group consisting of acetogenic and methanogenic.

6. The method of claim 1, wherein a solids retention time (SRT) of the anaerobic digester is greater than a hydraulic retention time (HRT).

7. The method of claim 6, wherein the HRT is about 24 hours or less.

8. The method of claim 6, wherein the HRT is about 12 hours or less.

9. The method of claim 1, wherein a stream with higher protein content than that of the influent as measured by weight of dry matter is recovered.

10. The method of claim 9, wherein the stream with the higher protein content is concentrated using a process chosen from the group consisting of evaporation and membrane filtration.

11. The method of claim 9, wherein the protein is precipitated from an effluent.

12. The method of claim 1, further including the step of removing at least some of the soluble compounds prior to or after said converting step.

13. The method of claim 1, wherein a stream with higher lipid content than the influent, as measured by weight of dry matter, is recovered.

14. The method of claim 1, wherein the concentration of one or more organic compounds is reduced by about 50% or more, the organic compounds selected from the group consisting of acetic acid, lactic acid, other organic acids, ethyl alcohol, other alcohols, glycerol, and combinations thereof.

15. A method of processing spent grains, including the steps of:
separating a first stream consisting of spent grains into a second stream and a third stream wherein the second stream contains a majority of suspended solids;
separating the third stream into a fourth stream and a fifth stream wherein the fifth stream is lower in suspended solids than the fourth stream;
directing the fifth stream to an anaerobic digester; and
converting at least some organic compounds to a biogas.

16. The method of claim 15, wherein the organic compounds include one or more compounds chosen from the group consisting of acetic acid, lactic acid, other organic acids, ethyl alcohol, other alcohols, glycerol, and combinations thereof

17. The method of claim 15, further including the step of removing additional soluble compounds from the second stream by combing the second stream with at least one diluent.

18. The method of claim 15, further including the step of removing additional soluble compounds from the fourth stream by combing the fourth stream with at least one diluent.

19. The method of claim 17, further including the step of recovering suspended solids from the fourth stream.

20. A method of fermenting a grain product, including the steps of:
mixing a grain product with a liquid to produce a slurry;
adding a microorganism to the slurry to produce a product of fermentation;
removing a product of fermentation from the slurry to produce spent grains; and
directing at least of portion of the spent grains to a anaerobic digester.

21. The method of claim 20, wherein the anaerobic digester contains microorganisms primarily classified chosen from a group consisting of acetogenic and methanogenic.

22. The method of claim 20, wherein the concentration of one or more organic compounds is reduced by about 50% or more, the organic compounds selected from the group consisting of acetic acid, lactic acid, other organic acids, ethyl alcohol, other alcohols, glycerol, and combinations thereof.

23. The method of claim 20, further including the step of using effluent of the anaerobic digester as a fermentation media.

24. The method of claim 20, further including the step of recycling effluent of the anaerobic digester to a fermentation process.

25. The method of claim 20, wherein effluent of the anaerobic digester is recovered.

26. The method of claim 25, wherein the recovered effluent is higher in protein that influent to the anaerobic digester.

27. The method of claim 25, further including at least one step chosen from the group consisting of dehydrating the effluent, drying the effluent, and combinations thereof.

28. The method of claim 25, further including the step of concentrating the effluent.

29. The method of claim 25, further including the step of adding at least some of the effluent to at least some of the spent grains.

30. The method of claim 20, further including the step of recovering bio-solids from the anaerobic digester.

31. The method of claim 30, further including at least one step chosen from the group consisting of dewatering the bio-solids, drying the bio-solids, and adding the bio-solids to the spent grains of a fermentation process or portions thereof.
